(19) United States
(12) Patent Application Publication
(10) Pub. No.: US 2007/0270736 A1
(43) Pub. Date: Nov. 22, 2007

(54) Bulb Syringe Nasal Protection Device

(76) Inventor: Rosana Giarrocco-Brettner, Dublin, CA (US)

Correspondence Address: GREENBERG & LIEBERMAN, LLC, 2141 WISCONSIN AVE, N.W., SUITE C-2, WASHINGTON, DC, 20007, US

(21) Appl. No.: 11/163,828

(22) Filed: Nov. 1, 2005

(51) Int. Cl. A61M 5/178; A62B 18/02

(52) U.S. Cl. 604/37; 128/207.13

(57) ABSTRACT

An improvement in bulb syringes which essentially prevents the bulb syringe from entering too far into the nasal cavity of a baby or infant when removing mucus from the child's nose, thereby allowing easier breathing for the child. The device is a guard member and is screwed onto the bulb syringe and is made of a soft plastic material. When the bulb syringe is used to suction, the guard member impedes the stem of the bulb syringe from going too far into the nasal passages of a baby, which reduces discomfort, anxiety and pain in the child. Generally the invention is applicable to any previously patented bulb syringe, no matter the shape or size of the syringe, and is a unique improvement on existing bulb syringes.

### FIELD OF THE INVENTION

The present invention relates to an improvement in the bulb syringe device for relieving babies and infants of congestion.

### BACKGROUND OF THE INVENTION

Occasionally a baby or infant will be congested and have difficulty breathing due to the buildup of mucus within the nose and nasal passages. An easy remedy is generally using a bulb syringe. It is generally an apparatus made of a rubber-like material, and it has a bulb, stem, and a thin tip opening; when the bulb is pressed down upon, the air inside the bulb is released, and when the hand releases pressure of the bulb, the air is sucked back into the bulb. It can also be used as a dropper for liquids or medication. It is essentially a small vacuum.

To help babies who are congested or have stuffy noses, the bulb syringe can be an effective solution. Before inserting the bulb into the baby's nose, one must press down upon it to release the air from the bulb (away from the baby's face). The opening is inserted into the nostril in order to remove mucus and clear the breathing passages of the nose. When the hand releases the bulb, the mucus is sucked into the syringe and removed from the baby's nostril. The bulb releases the mucus by pressing down on said bulb again (away from baby's face). The bulb syringe may also be used as a mouth syringe to remove fluids from the throat.

Often times, a syringe will have a long, thin stem, and may cause pain or discomfort to the baby, as the syringe may unintentionally be placed too far inside the nasal cavity. Some children may also resist use of the syringe because of the discomfort associated with the device. This makes it hard for parents or caretakers to use the bulb syringe to effectively suction the mucus from a child's nose.

Relevant art includes U.S. Pat. No. 3,635,218, issued to Ericson on Jan. 18, 1972. It describes a bulb-piston syringe similar to the one used in the present invention. However, this syringe has a long stem, which can easily be placed too far up the nasal cavity of the infant.

U.S. Pat. No. 3,651,808, issued to White on Mar. 28, 1972, and U.S. Pat. No. 4,258,714, issued to Leopoldi et al. on Mar. 31, 1981 disclose an ear syringe. Unlike the present invention, these two patents are intended for the ear and not the nose or mouth; additionally, they have a curvature that is not appropriate for the nasal canal.

U.S. Pat. No. 3,705,583, issued to Rentsch on Dec. 12, 1972, is a syringe assembly. It is intended primarily for medical use and is comprised of a bulb with a wide-end barrel (stem) meant to receive a liquid or medication.

U.S. Pat. No. 3,705,584, issued to Fript on Dec. 12, 1972, is a stabilized syringe assembly. It has a stabilizing device, which props the syringe up in order to avoid leakage of the contents in the barrel when the syringe is placed on a table or the like. It is also intended for medical usage and makes no reference to an additional device that prevents the syringe from going too far up the nasal cavity of a baby or an infant.

U.S. Pat. No. 4,973,250, issued to Milman on Nov. 27, 1990, is a periodontal syringe intended for the mouth and teeth (particularly for the irrigation and flushing of pockets in the gum due to the removal of wisdom teeth or the like). It is intended to carry a medication that is placed in the mouth, not to suction an infant's nose.

U.S. Pat. No. 5,758,802, issued to Wallays on Jun. 2, 1998, is an icing set. It is a syringe-like device that is used to decorate cakes and pastries. It is not intended for use on a person. U.S. Pat. No. 5,817,066, issued to Goforth on Oct. 6, 1998, is a bulb-type irrigation syringe intended for use as a feeding syringe in medical patients. U.S. Pat. No. 5,848,993, issued to Tanhehco et al. on Dec. 15, 1998, discloses a bulb syringe, which is transparent so that the user may see how much liquid is found within the bulb; it also comprises two finger-rests on the sides to facilitate gripping the bulb. It does not, however, account for the stem being accidentally placed too far in the infant's nasal passages.

U.S. Pat. No. 6,290,667, issued to Cook on Sep. 18, 2001, is a nasal aspirator. It is similar to the present invention in that it has a smaller stem and is in the shape of a teddy bear (which is more appealing to children). However, the aforementioned patent does not have a removable device that serves as a stopper, which provides the user with the possibility of having a mouth and nasal syringe in one device. The length of the stem is also not adjustable as it is in the present invention.

Various bulb syringes have been informally tested and found to be ineffective. Some may have a widened or shorter stem, and yet they may still go up too far in the nasal cavity. Over-insertion can still occur, especially if the child is struggling against it like an infant or toddler would. Additionally, a shorter stem is not as effective in the removal of fluids as a long stem is, and it cannot be used interchangeably as a nose or a mouth syringe. Some existing models are made of a hard plastic, which have been tested in actual use and found to be very uncomfortable for the baby. Of the relevant art, none have an add-on feature that would serve as a stopper and prevent over-insertion when using a long-stem bulb syringe in the nose.

Hence, there is a need for a device which will effectively clear mucus from a baby's nose in order to aid the child in breathing. Additionally, this device should not cause pain or discomfort to the baby by preventing the syringe from going up too far in the nasal cavity, and can be used as a mouth syringe if necessary.

It is expected that none of the relevant art can accomplish these objectives, as existing models of bulb syringes are inadequate or are primarily intended for other uses, and thus the present invention provides a simple and adequate solution to the existing problem.

### SUMMARY OF THE INVENTION

The present invention is an improvement upon existing bulb syringes. It is comprised of a bulb with a guard member, which is placed over the tip of the stem that prevents the bulb syringe from being placed too far in the nasal cavity, which can cause discomfort to the child. The guard member is made of a pliable but firm plastic that provides maximum comfort to the child. The stem of the bulb syringe has several ridges, which are used to screw on the guard member so that it will not fall off on its own. Either instructions on appropriate length will be included with the purchase of the present invention, or the guard member will be set at a predetermined length on the syringe.

The guard member is twisted onto the bulb syringe at the appropriate length. Air is squeezed out of the bulb syringe, pointed away from baby's face. The tip is gently inserted into the baby's nostril. Pressure is released and mucus is removed. The bulb syringe is removed and the mucus is discarded by squeezing the bulb again (away from baby's face). The process can then be repeated in the other nostril. The bulb syringe with the guard member can also be used to irrigate the nostrils.

It is an object of the present invention to improve upon existing bulb syringes to reduce the likelihood of discomfort, pain or injury in babies and infants when said syringe is used to relieve the child of congestion in the nose.

It is therefore an object of the present invention to have a guard member that is mounted on a bulb syringe and acts as a stopper in order to prevent the syringe from accidentally going too far up the nasal cavity of the child.

It is an object of the present invention, in one embodiment, to be used as a nose or a mouth syringe, with said guard member being removable. In another embodiment of the present invention the guard member is manufactured as a permanent attachment to the bulb syringe and is intended specifically for removal of mucus in the baby's nose.

It is an object of the present invention to make the bulb syringe comfortable for the child, thereby easing the baby's anxiety and facilitating the use of the syringe, which will help the child breathe easier. It is an object of the present invention to accomplish this without the necessity of medical intervention as the process can be done at home with a hand-held bulb syringe and attachment. Unlike existing syringes that are available to the public and may be used without proper safety precautions or supervision, the present invention prevents improper use of nasal aspirators. The present invention also aims to make suctioning the nose or mouth quick and easy for the parent or caretaker, and painless for the child.

It is an object of the present invention to prevent injury by controlling the depth of which the syringe enters the baby's nose. Furthermore, the device allows quick removal of fluids without worrying about causing pain or injury to the child. It provides for a tighter seal when suctioning, because the guard member covers the nostril momentarily and impedes air from escaping the nose when using the bulb syringe. The shape of the guard member itself, an oval and convex disc made of a soft plastic, provides comfort and security to the bulb syringe.

The guard member attached to the bulb syringe is as secure as possible. This is done through the use of ridges on the syringe and the guard member, and a screw-like motion secures the guard member on the bulb syringe.

It is a final object of the present invention to permit the syringe to be as sanitary and hygienic as possible. When the bulb is laid on its side (on a table or the like) the tip/opening of the syringe is prevented from touching surfaces because the guard member supports the stem, keeping it a few centimeters off of the surface.

### BRIEF DESCRIPTION OF THE DRAWINGS

FIG. 1 is a view of the present embodiment with the distinct parts (guard member and generic bulb syringe) unattached.

FIG. 2 is a second view of the present embodiment with the guard member on the syringe.

FIG. 3 is an environmental perspective of the present invention in use.

### DETAILED DESCRIPTION OF THE PREFERRED EMBODIMENT

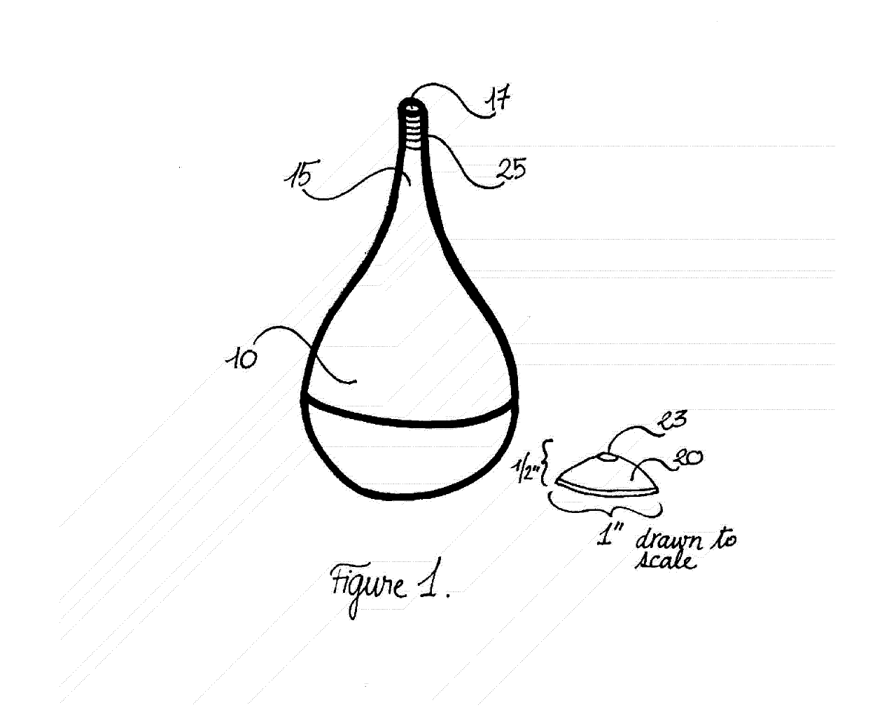

FIG. 1 shows a typical bulb syringe 10 with a stem 15 and opening 17. The guard member 20 is screwed onto the bulb syringe 10 by way of the ridges 25 on the stem 15 of the bulb 10. The guard member 20 is made of a soft but firm rubber material similar to the material used to manufacture the syringe 10. Preferably, it is approximately one inch in length and ½ inch in width, with a slightly rounded top. The guard member 20 has such preferred measurements as it must not be too large as to obstruct the proper placement of the opening 17 of the stem 15 in the nostril. Preferably, the guard member 20 is therefore oval-shaped, as the longer curves of the oval facilitate the placement of the stem against the region between the mouth and nose, and the stem can more easily penetrate the nostril while still providing protection. The guard member 20 has a hole 23 in the center, which also has ridges 25 so that the guard member 20 can be screwed onto the syringe 10 to adjust the length of the syringe stem 15 entering the nose. The guard member 20 is therefore removable if the syringe 10 needs to be used in the mouth.

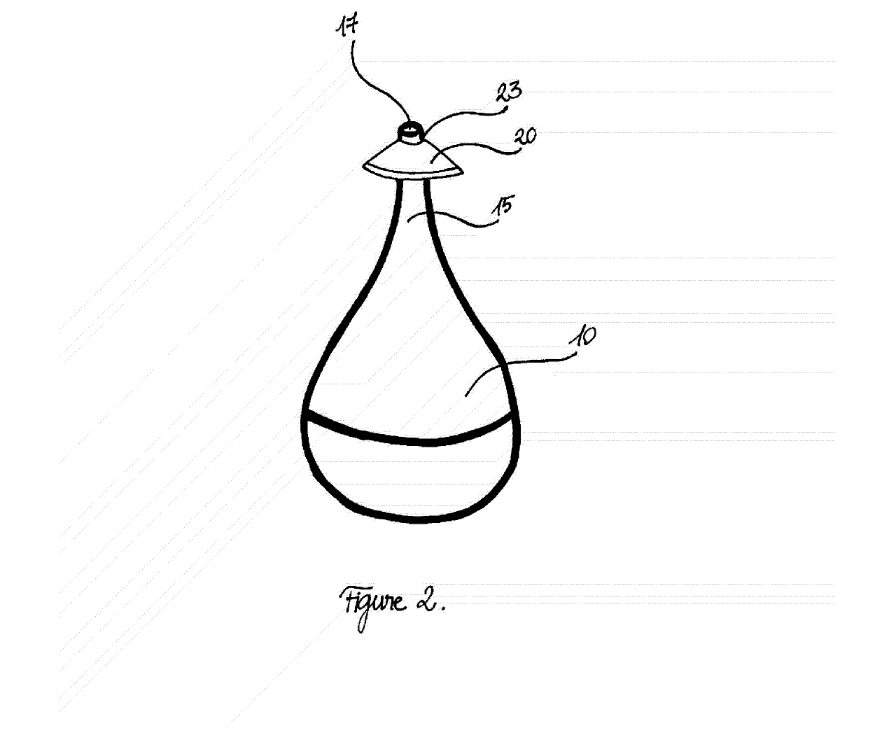

As shown in FIG. 2, the guard member 20, through the hole 23, is screwed into place on the stem 15 of the bulb 10. There are instructions that accompany the guard member 20 in order to ensure that it is set at the correct length on the bulb syringe 10, or it may be a predetermined length. The opening 17 of the bulb 10 is still exposed in order for the suctioning to take place. In an alternative embodiment of the present invention, the guard member 20 is manufactured to be a permanent structure of the bulb syringe 10, but in this case the bulb syringe 10 would only be used for the nose and not the mouth.

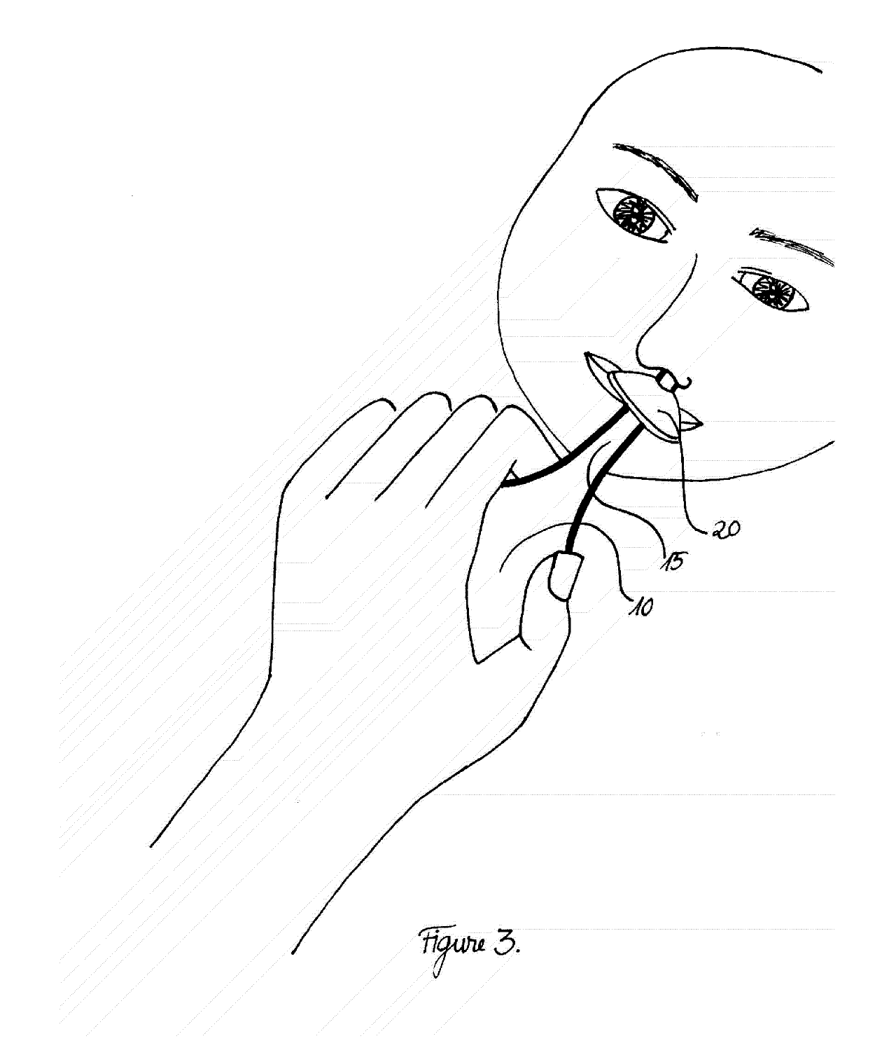

As shown in FIG. 3, the tip/opening 17 of the bulb syringe 10 is inserted in the nose of the infant and the suctioning takes place. The guard member 20 prevents the stem 15 of the bulb 10 from going too far in the nasal cavity and prevents any accidental injury.

It should be understood that there might be variations in the present invention that are not limited to the detailed description of the embodiment, but still capture the essence of the invention as dictated in the following claims.

I claim:

1. A nasal protection device, comprising:
a bulb syringe having a bulb and an elongated stem; and
a guard member that is constructed to attach onto said stem of said bulb syringe, said guard member having a concave shape that curves towards said bulb.

2. The nasal protection device of claim 1 wherein said guard member is joined to said stem of said bulb syringe, perpendicular to the axial plane of said stem.

3. The nasal protection device of claim 2, wherein said guard member is oval in shape, with a slightly rounded top.

4. The nasal protection device of claim 3, wherein said guard member that measures approximately at least one inch in length or ½ inch in width.

5. The nasal protection device of claim 1, wherein said guard member serves as a nasal protection device when employing said bulb syringe to suction an infant's nose, thereby preventing injury, discomfort, or pain to a child.

6. The nasal protection device of claim 1, wherein said guard member is removable from said bulb syringe, and said bulb syringe can additionally be employed as a mouth suction.

7. The nasal protection device of claim 1, wherein said guard member has a centrally placed hole configured to receive said elongated stem.

8. The nasal protection device of claim 7 wherein said centrally placed hole has small ridges on its interior allowing said guard member to be in communication with said bulb syringe.

9. The nasal protection device of claim 1, wherein said guard member is configured to control the depth of insertion of said elongated stem.

10. The nasal protection device of claim 9, wherein said guard member is configured to tighten the seal between a nostril of a child and said stem.

11. The nasal protection device of claim 1 wherein said guard member is configured to prevent said stem from touching a flat surface when said bulb syringe is laid in a horizontal fashion on a flat surface.

12. The nasal protection device of claim 1 wherein said guard member is configured to be molded permanently onto said stem.

13. The nasal protection devise of claim 1, wherein said stem has a first end, a second end and a mid section, said first end in communication with said bulb, said second end configured to terminate in a cylindrical shape, said mid section having a cylindrical shape, and said guard member covering only said second end.
